(19) United States
(12) Patent Application Publication
(10) Pub. No.: US 2007/0032812 A1
(43) Pub. Date: Feb. 8, 2007

(54) Method and apparatus for a tissue penetrating device user interface

(75) Inventors: Dieter Loerwald, Hamburg (DE); Dirk Boecker, Palo Alto, CA (US); Don Alden, Sunnyvale, CA (US); Dominique Freeman, La Honda, CA (US)

Correspondence Address: HELLER EHRMAN LLP, 275 MIDDLEFIELD ROAD, MENLO PARK, CA, 94025-3506, US

(73) Assignee: Pelikan Technologies, inc.

(21) Appl. No.: 10/555,249

(22) PCT Filed: May 3, 2004
(86) PCT No.: PCT/US04/13856

§ 371 Date: Jul. 28, 2006

Related U.S. Application Data
(60) Provisional application No. 60/467,680, filed on May 2, 2003.

(51) Int. Cl. A61B 17/32

(52) U.S. Cl. 606/181

(57) ABSTRACT

A tissue penetrating system is provided. The system may include a housing, a penetrating members positioned in the housing, and a visual display on the housing, the visual display having at lease one visual indicator positioned next to a corresponding marking on the housing. In one embodiment, the visual display may provide a variety of information about the function and performance of the penetrating member.

### BACKGROUND OF THE INVENTION

Lancing devices are known in the medical health-care products industry for piercing the skin to produce blood for analysis. Typically, a drop of blood for this type of analysis is obtained by making a small incision in the fingertip, creating a small wound, which generates a small blood droplet on the surface of the skin.

Early methods of lancing included piercing or slicing the skin with a needle or razor. Current methods utilize lancing devices that contain a multitude of spring, cam and mass actuators to drive the lancet. These include cantilever springs, diaphragms, coil springs, as well as gravity plumbs used to drive the lancet. The device may be held against the skin and mechanically triggered to ballistically launch the lancet. Unfortunately, the pain associated with each lancing event using known technology discourages patients from testing. In addition to vibratory stimulation of the skin as the driver impacts the end of a launcher stop, known spring based devices have the possibility of harmonically oscillating against the patient tissue, causing multiple strikes due to recoil. This recoil and multiple strikes of the lancet against the patient is one major impediment to patient compliance with a structured glucose monitoring regime.

Another impediment to patient compliance is the lack of spontaneous blood flow generated by known lancing technology. In addition to the pain as discussed above, a patient may need more than one lancing event to obtain a blood sample since spontaneous blood generation is unreliable using known lancing technology. Thus the pain is multiplied by the number of tries it takes to successfully generate spontaneous blood flow. Different skin thickness may yield different results in terms of pain perception, blood yield and success rate of obtaining blood between different users of the lancing device. Known devices poorly account for these skin thickness variations.

A still further impediment to improved compliance with glucose monitoring are the many steps and hassle associated with each lancing event. Many diabetic patients that are insulin dependent may need to self-test for blood glucose levels five to six times daily. The large number of steps required in traditional methods of glucose testing, ranging from lancing, to milking of blood, applying blood to the test strip, and getting the measurements from the test strip, discourages many diabetic patients from testing their blood glucose levels as often as recommended. Older patients and those with deteriorating motor skills encounter difficulty loading lancets into launcher devices, transferring blood onto a test strip, or inserting thin test strips into slots on glucose measurement meters. Additionally, the wound channel left on the patient by known systems may also be of a size that discourages those who are active with their hands or who are worried about healing of those wound channels from testing their glucose levels.

### SUMMARY OF THE INVENTION

Accordingly, an object of the present invention is to provide improved tissue penetrating systems, and their methods of use.

These and other objects of the present invention are achieved in a skin penetrating system including a penetrating member positioned in a housing member. A tissue stabilizing device may be coupled to the housing member.

In one embodiment of the present invention, a tissue penetrating system is provided. The system may include a housing, a penetrating members positioned in the housing, and a visual display on the housing, wherein the visual display has at lease one visual indicator position next to a corresponding marking on the housing.

In another embodiment of the present invention, another tissue penetrating system is provided comprising a housing, a penetrating members positioned in the housing, a tissue pressure applicator coupled to the housing member, and a visual display on the housing, wherein the visual display has at lease one visual indicator position next to a corresponding marking on the housing.

In a still further embodiment of the present invention, another tissue penetrating system is provided. The system comprises a housing, a penetrating members positioned in the housing, an analyte detecting member coupled to a sample chamber, the analyte detecting member being configured to determine a concentration of an analyte in a body fluid using a sample of less than 1 microliter (μL) of a body fluid disposed in the sample chamber; and a visual display on the housing, the visual display having a screen saver which is activated after a period of non-use by a user. The period of non-use may be preset or it can be set by the user.

In yet another embodiment of the present invention, another tissue penetrating system is provided. The system comprises a housing, a penetrating members positioned in the housing, a visual display on the housing, the visual display having at lease one visual indicator position next to a corresponding marking on the housing, and a series of buttons on the housing for changing penetrating member settings shown on the visual display.

A further understanding of the nature and advantages of the invention will become apparent by reference to the remaining portions of the specification and drawings.

### BRIEF DESCRIPTION OF THE DRAWINGS

FIG. 1 illustrates an embodiment of a controllable force driver in the form of a cylindrical electric penetrating member driver using a coiled solenoid-type configuration.

FIG. 2A illustrates a displacement over time profile of a penetrating member driven by a harmonic spring/mass system.

FIG. 2B illustrates the velocity over time profile of a penetrating member driver by a harmonic spring/mass system.

FIG. 2C illustrates a displacement over time profile of an embodiment of a controllable force driver.

FIG. 2D illustrates a velocity over time profile of an embodiment of a controllable force driver.

FIG. 3 is a diagrammatic view illustrating a controlled feed-back loop.

FIG. 4 is a perspective view of a tissue penetration device having features of the invention.

FIG. 5 is an elevation view in partial longitudinal section of the tissue penetration device of FIG. 4.

FIG. 6 is a front view of one embodiment of a lancing device according to the present invention.

FIG. 7 shows a side view of the device of FIG. 6.

FIG. 8 shows a perspective view of the device of FIG. 6.

FIG. 9 shows a bottom view of the device of FIG. 6.

FIGS. 10 and 11 show perspective views of one embodiment of the lancing device.

FIG. 12 shows a front view of one embodiment of a device according to the present invention.

FIG. 13 shows icons and text that can be displayed on one embodiment of a display according to the present invention.

FIG. 14 shows a display that includes one embodiment of a screen saver according to the present invention.

FIGS. 15-17 show icons that can appear on embodiments of a display according to the present invention.

FIG. 18 shows one embodiment of a cartridge containing a plurality of penetrating members.

FIG. 19 shows another embodiment of a cartridge containing a plurality of penetrating members.

FIGS. 20 and 21 show another embodiment of a cartridge containing a plurality of penetrating members.

FIG. 22 shows a perspective view of a bandolier-type device using a tape holding a plurality of penetrating members.

### DESCRIPTION OF THE SPECIFIC EMBODIMENTS

The present invention provides a solution for body fluid sampling. Specifically, some embodiments of the present invention provides a penetrating member device for consistently creating a wound with spontaneous body fluid flow from a patient. The invention may be a multiple penetrating member device with an optional high density design. It may use penetrating members of smaller-size than-known penetrating members. The-device may be-used for multiple lancing events without having to remove a disposable from the device or for the user to handle sharps. The invention may provide improved sensing capabilities. At least some of these and other objectives described herein will be met by embodiments of the present invention.

It is to be understood that both the foregoing general description and the following detailed description are exemplary and explanatory only and are not restrictive of the invention, as claimed. It should be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

“Analyte detecting member” refers to any use, singly or in combination, of chemical test reagents and methods, electrical test circuits and methods, physical test components and methods, optical test components and methods, and biological test reagents and methods to yield information about a blood sample. Such methods are well known in the art and may be based on teachings of, e.g. Tietz Textbook of Clinical Chemistry, 3d Ed., Sec. V, pp. 776-78 (Burtis & Ashwood, Eds., W.B. Saunders Company, Philadelphia, 1999); U.S. Pat. No. 5,997,817 to Chrismore et al. (Dec. 7, 1999); U.S. Pat. No. 5,059,394 to Phillips et al. (Oct. 22, 1991); U.S. Pat. No. 5,001,054 to Wagner et al. (Mar. 19, 1991); and U.S. Pat. No. 4,392,933 to Nakamura et al. (Jul. 12, 1983), the teachings of which are hereby incorporated by reference, as well as others. Analyte detecting member may include tests in the sample test chamber that test electrochemical properties of the blood, or they may-include optical means for sensing optical properties of the blood (e.g. oxygen saturation level), or they may include biochemical reagents (e.g. antibodies) to sense properties (e.g. presence of antigens) of the blood. The analyte detecting member may comprise biosensing or reagent material that will react with an analyte in blood (e.g. glucose) or other body fluid so that an appropriate signal correlating with the presence of the analyte is generated and can be read by the reader apparatus. By way of example and not limitation, analyte detecting member may “associated with”, “mounted within”, or “coupled to” a chamber or other structure when the analyte detecting member participates in the function of providing an appropriate signal about the blood sample to the reader device. Analyte detecting member may also include nanowire analyte detecting members as described herein. Analyte detecting member may use potentiometric, coulometric, or other method useful for detection of analyte levels.

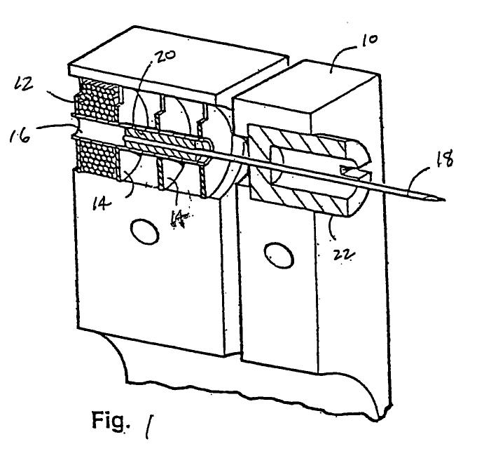

The present invention may be used with a variety of different penetrating member drivers. It is contemplated that these penetrating member drivers may be spring based, solenoid based, magnetic driver based, nanomuscle based, or based on any other mechanism useful in moving a penetrating member along a path into tissue. It should be noted that the present invention is not limited by the type of driver used with the penetrating member feed mechanism. One suitable penetrating member driver for use with the present invention is shown in FIG. 1. This is an embodiment of a solenoid type electromagnetic driver that is capable of driving an iron core or slug mounted to the penetrating member assembly using a direct current (DC) power supply. The electromagnetic driver includes a driver coil pack that is divided into three separate coils along the path of the penetrating member, two end coils and a middle coil. Direct current is alternated to the coils to advance and retract the penetrating member. Although the driver coil pack is shown with three coils, any suitable number of coils may be used, for example, 4, 5, 6, 7 or more coils may be used.

Referring to the embodiment of FIG. 1, the stationary iron housing 10 may contain the driver coil pack with a first coil 12 flanked by iron spacers 14 which concentrate the magnetic flux at the inner diameter creating magnetic poles. The inner insulating housing 16 isolates the penetrating member 18 and iron core 20 from the coils and provides a smooth, low friction guide surface. The penetrating member guide 22 further centers the penetrating member 18 and iron core 20. The penetrating member 18 is protracted and retracted by alternating the current between the first coil 12, the middle coil, and the third coil to attract the iron core 20. Reversing the coil sequence and attracting the core and penetrating member back into the housing retracts the penetrating member. The penetrating member guide 22 also serves as a stop for the iron core 20 mounted to the penetrating member 18.

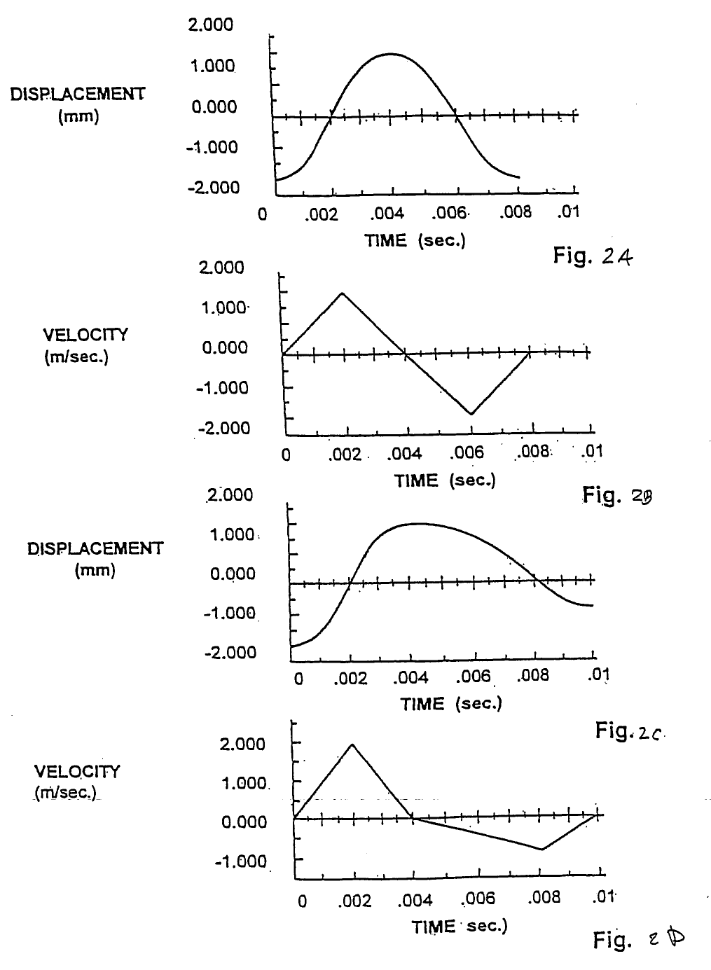

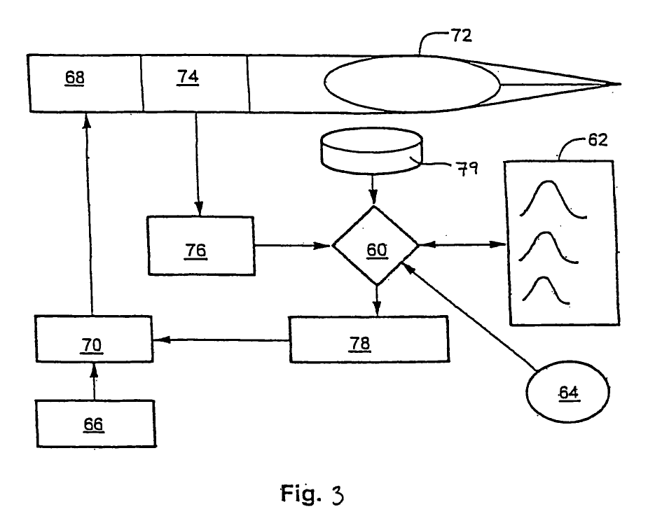

As discussed above, tissue penetration devices which employ spring or cam driving methods have a symmetrical or nearly symmetrical actuation displacement and velocity profiles on the advancement and retraction of the penetrating member as shown in FIGS. 2 and 3. In most of the available lancet devices, once the launch is initiated, the stored energy determines the velocity profile until the energy is dissipated. Controlling impact, retraction velocity, and dwell time of the penetrating member within the tissue can be useful in order to achieve a high success rate while accommodating variations in skin properties and minimize pain. Advantages can be achieved by taking into account of the fact that tissue dwell time is related to the amount of skin deformation as the penetrating member tries to puncture the surface of the skin and variance in skin deformation from patient to patient based on skin hydration.

In this embodiment, the ability to control velocity and depth of penetration may be achieved by use of a controllable force driver where feedback is an integral part of driver control. Such drivers can control either metal or polymeric penetrating members or any other type of tissue penetration element. The dynamic control of such a driver is illustrated in FIG. 2C which illustrates an embodiment of a controlled displacement profile and FIG. 2D which illustrates an embodiment of a the controlled velocity profile. These are compared to FIGS. 2A and 2B, which illustrate embodiments of displacement and velocity profiles, respectively, of a harmonic spring/mass powered driver. Reduced pain can be achieved by using impact velocities of greater than about 2 m/s entry of a tissue penetrating element, such as a lancet, into tissue. Other suitable embodiments of the penetrating member driver are described in commonly assigned, copending U.S. patent application Ser. No. 10/127,395, (Attorney Docket No. 38187-2551) filed Apr. 19, 2002 and previously incorporated herein.

FIG. 3 illustrates the operation of a feedback loop using a processor 60. The processor 60 stores profiles 62 in non-volatile memory. A user inputs information 64 about the desired circumstances or parameters for a lancing event. The processor 60 selects a driver profile 62 from a set of alternative driver profiles that have been preprogrammed in the processor 60 based on typical or desired tissue penetration device performance determined through testing at the factory or as programmed in by the operator. The processor 60 may customize by either scaling or modifying the profile based on additional user input information 64. Once the processor has chosen and customized the profile, the processor 60 is ready to modulate the power from the power supply 66 to the penetrating member driver 68 through an amplifier 70. The processor 60 may measure the location of the penetrating member 72 using a position sensing mechanism 74 through an analog to digital converter 76 linear encoder or other such transducer. Examples of position sensing mechanisms have been described in the embodiments above and may be found in the specification for commonly assigned, copending U.S. patent application Ser. No. 10/127,395, (Attorney Docket No. 38187-2551) filed Apr. 19, 2002 and previously incorporated herein. The processor 60 calculates the movement of the penetrating member by comparing the actual profile of the penetrating member to the predetermined profile. The processor 60 modulates the power to the penetrating member driver 68 through a signal generator 78, which may control the amplifier 70 so that the actual velocity profile of the penetrating member does not exceed the predetermined profile by more than a preset error limit. The error limit is the accuracy in the control of the penetrating member.

After the lancing event, the processor 60 can allow the user to rank the results of the lancing event. The processor 60 stores these results and constructs a database 80 for the individual user. Using the database 79, the processor 60 calculates the profile traits such as degree of painlessness, success rate, and blood volume for various profiles 62 depending on user input information 64 to optimize the profile to the individual user for subsequent lancing cycles. These profile traits depend on the characteristic phases of penetrating member advancement and retraction. The processor 60 uses these calculations to optimize profiles 62 for each user. In addition to user input information 64, an internal clock allows storage in the database 79 of information such as the time of day to generate a time stamp for the lancing event and the time between lancing events to anticipate the user's diurnal needs. The database stores information and statistics for each user and each profile that particular user uses.

In addition to varying the profiles, the processor 60 can be used to calculate the appropriate penetrating member diameter and geometry suitable to realize the blood volume required by the user. For example, if the user requires about 1-5 microliter volume of blood, the processor 60 may select a 200 micron diameter penetrating member to achieve these results. For each class of lancet, both diameter and lancet tip geometry, is stored in the processor 60 to correspond with upper and lower limits of attainable blood volume based on the predetermined displacement and velocity profiles.

The lancing device is capable of prompting the user for information at the beginning and the end of the lancing event to more adequately suit the user. The goal is to either change to a different profile or modify an existing profile. Once the profile is set, the force driving the penetrating member is varied during advancement and retraction to follow the profile. The method of lancing using the lancing device comprises selecting a profile, lancing according to the selected profile, determining lancing profile traits for each characteristic phase of the lancing cycle, and optimizing profile traits for subsequent lancing events.

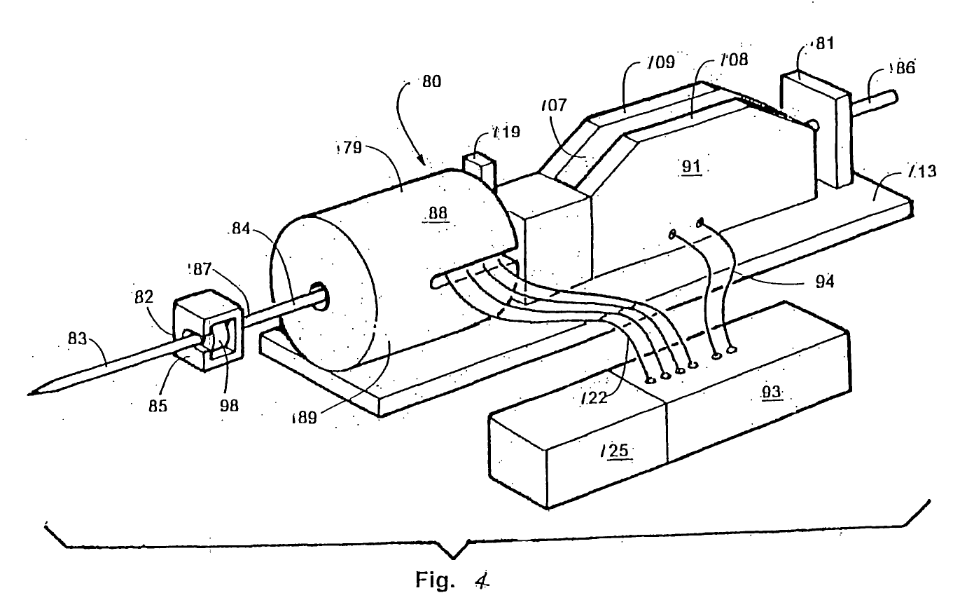

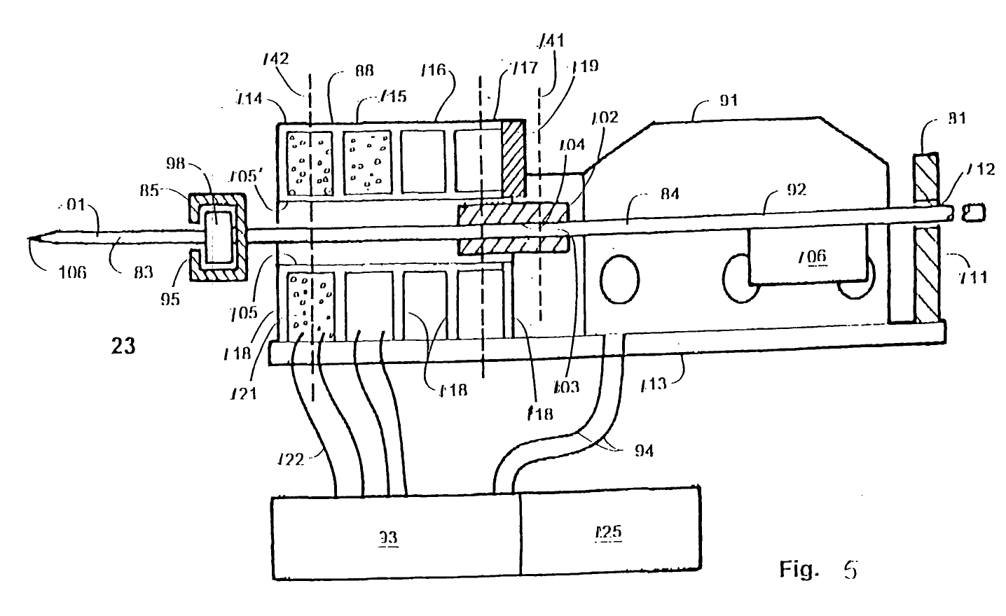

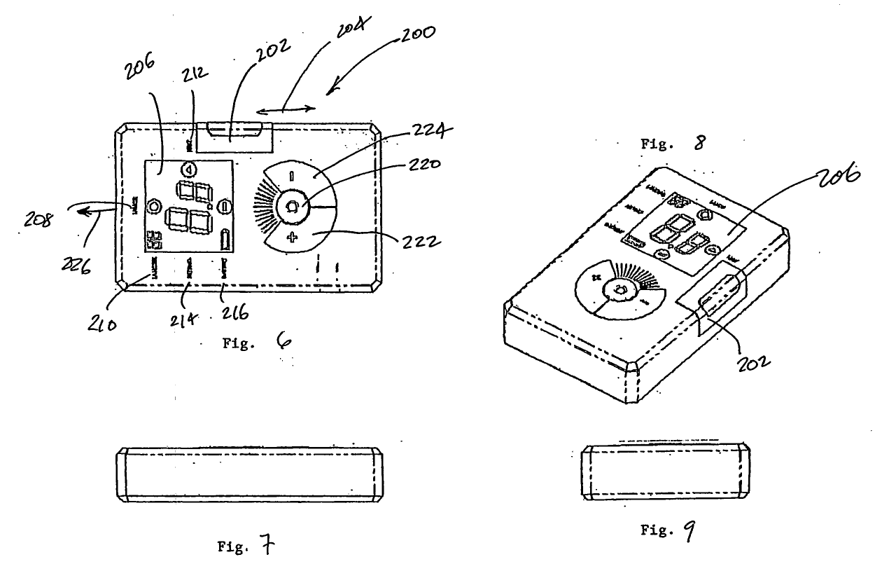

FIG. 4 illustrates an embodiment of a tissue penetration device, more specifically, a lancing device 80 that includes a controllable driver 179 coupled to a tissue penetration element. The lancing device 80 has a proximal end 81 and a distal end 82. At the distal end 82 is the tissue penetration element in the form of a penetrating member 83, which is coupled to an elongate coupler shaft 84 by a drive coupler 85. The elongate coupler shaft 84 has a proximal end 86 and a distal end 87. A driver coil pack 88 is disposed about the elongate coupler shaft 84 proximal of the penetrating member 83. A position sensor 91 is disposed about a proximal portion 92 of the elongate coupler shaft 84 and an electrical conductor 94 electrically couples a processor 93 to the position sensor 91. The elongate coupler shaft 84 driven by the driver coil pack 88 controlled by the position sensor 91 and processor 93 form the controllable driver, specifically, a controllable electromagnetic driver.

Referring to FIG. 5, the lancing device 80 can be seen in more detail, in partial longitudinal section. The penetrating member 83 has a proximal end 95 and a distal end 96 with a sharpened point at the distal end 96 of the penetrating member 83 and a drive head 98 disposed at the proximal end 95 of the penetrating member 83. A penetrating member shaft 201 is disposed between the drive head 98 and the sharpened point 97. The penetrating member shaft 201 may be comprised of stainless steel, or any other suitable material or alloy and have a transverse dimension of about 0.1 to about 0.4 mm. The penetrating member shaft may have a length of about 3 mm to about 50 mm, specifically, about 15 mm to about 20 mm. The drive head 98 of the penetrating member 83 is an enlarged portion having a transverse dimension greater than a transverse dimension of the penetrating member shaft 201 distal of the drive head 98. This configuration allows the drive head 98 to be mechanically captured by the drive coupler 85. The drive head 98 may have a transverse dimension of about 0.5 to about 2 mm.

A magnetic member 102 is secured to the elongate coupler shaft 84 proximal of the drive coupler 85 on a distal portion 203 of the elongate coupler shaft 84. The magnetic member 102 is a substantially cylindrical piece of magnetic material having an axial lumen 204 extending the length of the magnetic member 102. The magnetic member 102 has an outer transverse dimension that allows the magnetic member 102 to slide easily within an axial lumen 105 of a low friction, possibly lubricious, polymer guide tube 105′ disposed within the driver coil pack 88. The magnetic member 102 may have an outer transverse dimension of about 1.0 to about 5.0 mm, specifically, about 2.3 to about 2.5 mm. The magnetic member 102 may have a length of about 3.0 to about 5.0 mm, specifically, about 4.7 to about 4.9 mm. The magnetic member 102 can be made from a variety of magnetic materials including ferrous metals such as ferrous steel, iron, ferrite, or the like. The magnetic member 102 may be secured to the distal portion 203 of the elongate coupler shaft 84 by a variety of methods including adhesive or epoxy bonding, welding, crimping or any other suitable method.

Proximal of the magnetic member 102, an optical encoder flag 206 is secured to the elongate coupler shaft 84. The optical encoder flag 206 is configured to move within a slot 107 in the position sensor 91. The slot 107 of the position sensor 91 is formed between a first body portion 108 and a second body portion 109 of the position sensor 91. The slot 107 may have separation width of about 1.5 to about 2.0 mm. The optical encoder flag 206 can have a length of about 14 to about 18 mm, a width of about 3 to about 5 mm and a thickness of about 0.04 to about 0.06 mm.

The optical encoder flag 206 interacts with various optical beams generated by LEDs disposed on or in the position sensor body portions 108 and 109 in a predetermined manner. The interaction of the optical beams generated by the LEDs of the position sensor 91 generates a signal that indicates the longitudinal position of the optical flag 206 relative to the position sensor 91 with a substantially high degree of resolution. The resolution of the position sensor 91 may be about 200 to about 400 cycles per inch, specifically, about 350 to about 370 cycles per inch. The position sensor 91 may have a speed response time (position/time resolution) of 0 to about 120,000 Hz, where one dark and light stripe of the flag constitutes one Hertz, or cycle per second. The position of the optical encoder flag 206 relative to the magnetic member 102, driver coil pack 88 and position sensor 91 is such that the optical encoder 91 can provide precise positional information about the penetrating member 83 over the entire length of the penetrating member's power stroke.

An optical encoder that is suitable for the position sensor 91 is a linear optical incremental encoder, model HEDS 9200, manufactured by Agilent Technologies. The model HEDS 9200 may have a length of about 20 to about 30 mm, a width of about 8 to about 12 mm, and a height of about 9 to about 11 mm. Although the position sensor 91 illustrated is a linear optical incremental encoder, other suitable position sensor embodiments could be used, provided they posses the requisite positional resolution and time response. The HEDS 9200 is a two channel device where the channels are 90 degrees out of phase with each other. This results in a resolution of four times the basic cycle of the flag. These quadrature outputs make it possible for the processor to determine the direction of penetrating member travel. Other suitable position sensors include capacitive encoders, analog reflective sensors, such as the reflective position sensor discussed above, and the like.

A coupler shaft guide 111 is disposed towards the proximal end 81 of the lancing device 80. The guide 111 has a guide lumen 112 disposed in the guide 111 to slidingly accept the proximal portion 92 of the elongate coupler shaft 84. The guide 111 keeps the elongate coupler shaft 84 centered horizontally and vertically in the slot 102 of the optical encoder 91.

The driver coil pack 88, position sensor 91 and coupler shaft guide 111 are all secured to a base 113. The base 113 is longitudinally coextensive with the driver coil pack 88, position sensor 91 and coupler shaft guide 111. The base 113 can take the form of a rectangular piece of metal or polymer, or may be a more elaborate housing with recesses, which are configured to accept the various components of the lancing device 80.

As discussed above, the magnetic member 102 is configured to slide within an axial lumen 105 of the driver coil pack 88. The driver coil pack 88 includes a most distal first coil 114, a second coil 115, which is axially disposed between the first coil 114 and a third coil 116, and a proximal-most fourth coil 117. Each of the first coil 114, second coil 115, third coil 116 and fourth coil 117 has an axial lumen. The axial lumens of the first through fourth coils are configured to be coaxial with the axial lumens of the other coils and together form the axial lumen 105 of the driver coil pack 88 as a whole. Axially adjacent each of the coils 114-117 is a magnetic disk or washer 118 that augments completion of the magnetic circuit of the coils 114-117 during a lancing cycle of the device 80. The magnetic washers 118 of the embodiment of FIG. 5 are made of ferrous steel but could be made of any other suitable magnetic material, such as iron or ferrite. The outer shell 89 of the driver coil pack 88 is also made of iron or steel to complete the magnetic path around the coils and between the washers 118. The magnetic washers 118 have an outer diameter commensurate with an outer diameter of the driver coil pack 88 of about 4.0 to about 8.0 mm. The magnetic washers 118 have an axial thickness of about 0.05, to about 0.4 mm, specifically, about 0.15 to about 0.25 mm.

Wrapping or winding an elongate electrical conductor 121 about an axial lumen until a sufficient number of windings have been achieved forms the coils 114-117. The elongate electrical conductor 121 is generally an insulated solid copper wire with a small outer transverse dimension of about 0.06 mm to about 0.88 mm, specifically, about 0.3 mm to about 0.5 mm. In one embodiment, 32 gauge copper wire is used for the coils 114-117. The number of windings for each of the coils 114-117 of the driver pack 88 may vary with the size of the coil, but for some embodiments each coil 114-117 may have about 30 to about 80 turns, specifically, about 50 to about 60 turns. Each coil 114-117 can have an axial length of about 1.0 to about 3.0 mm, specifically, about 1.8 to about 2.0 mm. Each coil 114-117 can have an outer transverse dimension or diameter of about 4.0, to about 2.0 mm, specifically, about 9.0 to about 12.0 mm. The axial lumen 105 can have a transverse dimension of about 1.0 to about 3.0 mm.

It may be advantageous in some driver coil 88 embodiments to replace one or more of the coils with permanent magnets, which produce a magnetic field similar to that of the coils when the coils are activated. In particular, it may be desirable in some embodiments to replace the second coil 115, the third coil 116 or both with permanent magnets. In addition, it may be advantageous to position a permanent magnet at or near the proximal end of the coil driver pack in order to provide fixed magnet zeroing function for the magnetic member (Adams magnetic Products 23A0002-flexible magnet material (800) 747-7543).

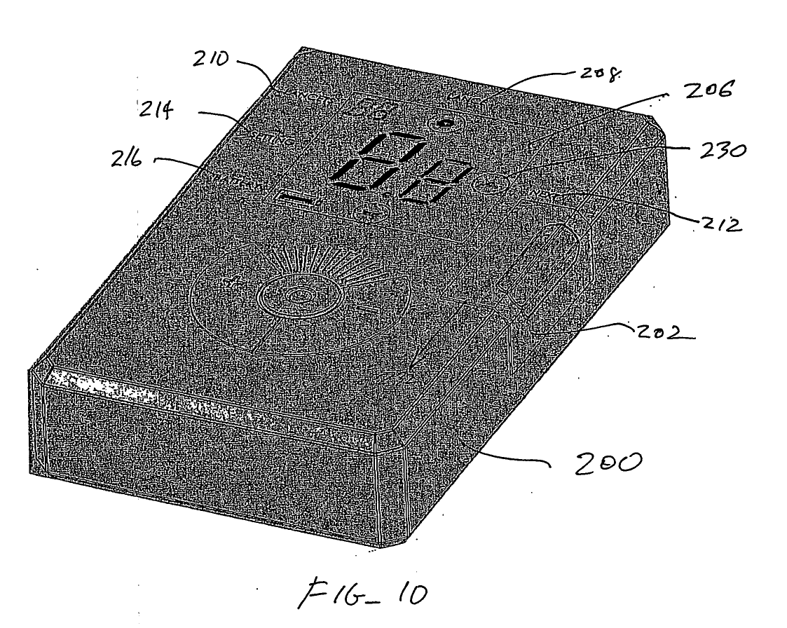

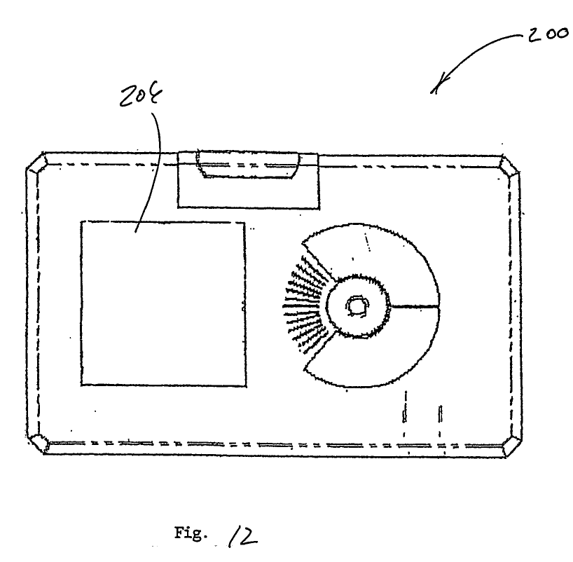

Referring now to the embodiment shown in FIGS. 6 through 9, various view of a housing 200 according to the present invention will now be described. FIG. 6 is a front view of the housing 200. The housing 200 includes a slide 202 which is movable as indicated by arrow 204. A visual display 206 may be included on the housing 200. The display 206 may function as a user interface and have indicators that correspond to markings 208, 210, 212, 214, and 216 on the housing 200. These indicators may be, but are not limited to, icons, numbers, words, colors, shapes, or other visual cue that may be displayed, flashed, faded, moved, or animated to communicate information to the user. At least one button 220 may also be included on the housing. A second button 222 and a third button 224. As seen in FIG. 6, the buttons 222 and 224 may have markings to provide an indication of their use. In one embodiment, the buttons 222 and 224 may be used to adjust lancing or penetrating member performance, such as but not limited to, lancing or penetrating member depth, lancing or penetrating member speed, dwell time, or any other lancing parameter as discussed herein. Button 220 may be used for actuating the penetrating member in the direction indicated by arrow 226 to create a wound in tissue. It should be understood that the buttons may have a variety of shapes such as round, triangular, rectangular, oval, square, polygonal, hexagonal, or any combination of the above. The device may have a single button or a plurality of buttons. The device may include a joystick or a multiple use button such as those found on some remotes for audio/video equipment, that allow a single button to provide multiple functionality and respond to be being pushed or directed in a variety of directions.

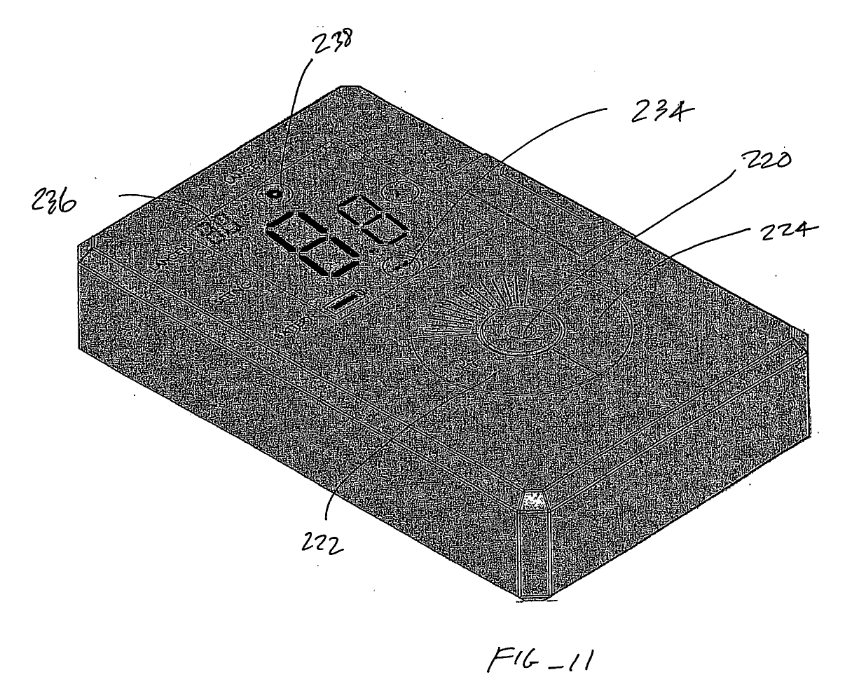

FIGS. 7, 8, and 9 show other views of the housing 200.

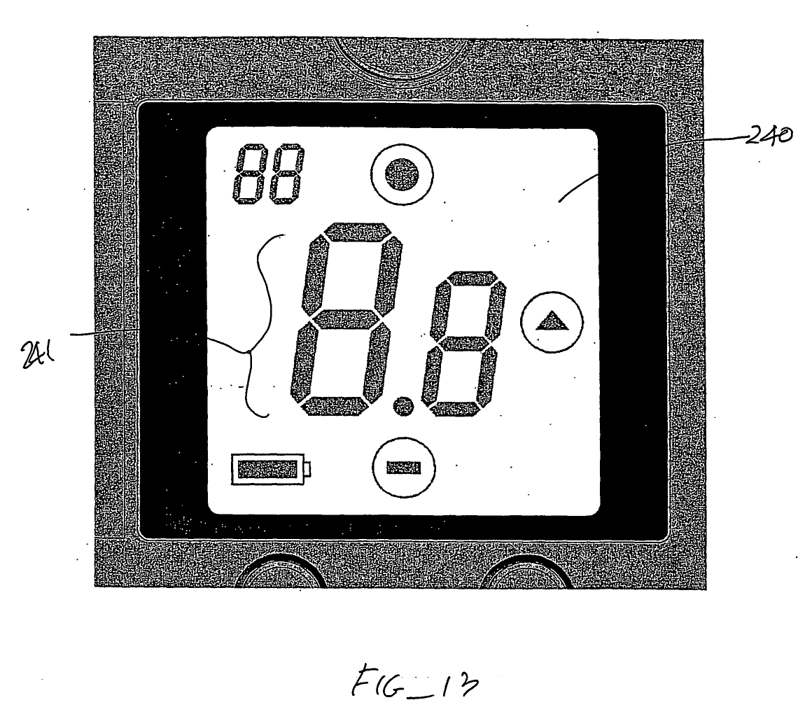

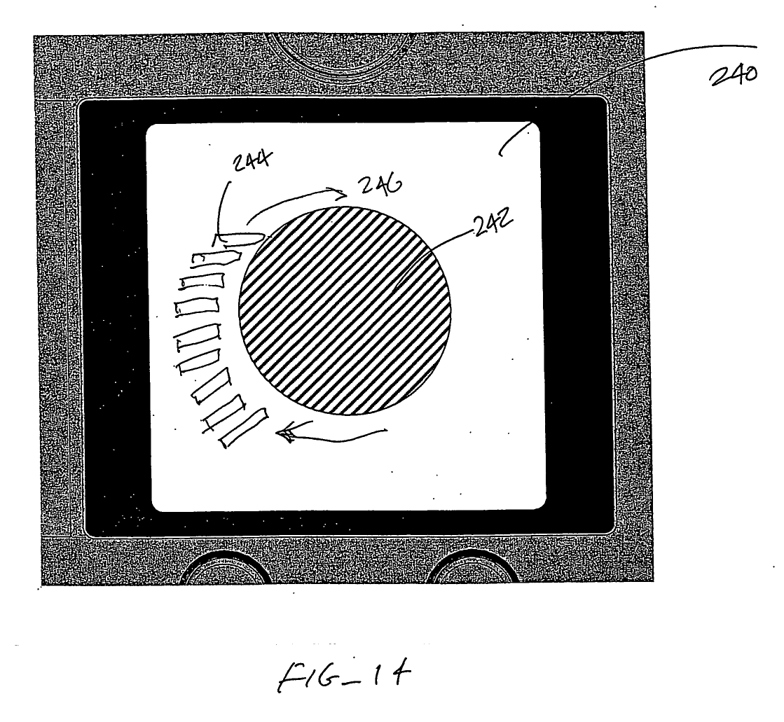

Referring now to FIGS. 10 and 11, still further views of an embodiment of the present invention is shown. In FIG. 10, the housing 200 is shown with markings 208, 210, 212, 214, and 216. Each marking may correspond with some type of visual indicator on display 206. This combination of markings (which may be permanent) on the housing 200 gives a user a greater sense of security or comfort in using the lancing device. The indicators on the display 206 may be placed next to, adjacent, or in proximity to the various markings associated with the indicators. As a nonlimiting example, the circled arrow indicator 230 will flash when the user needs to move the slider 202 as indicated by arrow 232. In some embodiments, the arrow 230 will move in the direction that the slider should be moved.

As seen in FIG. 11, when an lancing performance setting should be changed, a plus or minus sign 234 will be shown. A user may then adjust the performance setting by pressing buttons 222 or 224 as appropriate. The number of penetrating members or lancets may be displayed by indicator 236. When it is time to actuate the lancet for fluid sampling, an indicator 238 may appear. The indicator may be in the shape of the button 220. In other embodiments, it may be text, the shape of a lancet, an arrow, or other indicator associated with lancing.

Referring now to FIG. 12, an embodiment of the present invention is shown without markings on the housing 200. In this embodiment, the visual indicators will appear on the display 206. In a nonlimiting example, the display 206 may be an LCD display, a backlit display, a LCOS display, or a device for displaying icons and/or numerals.

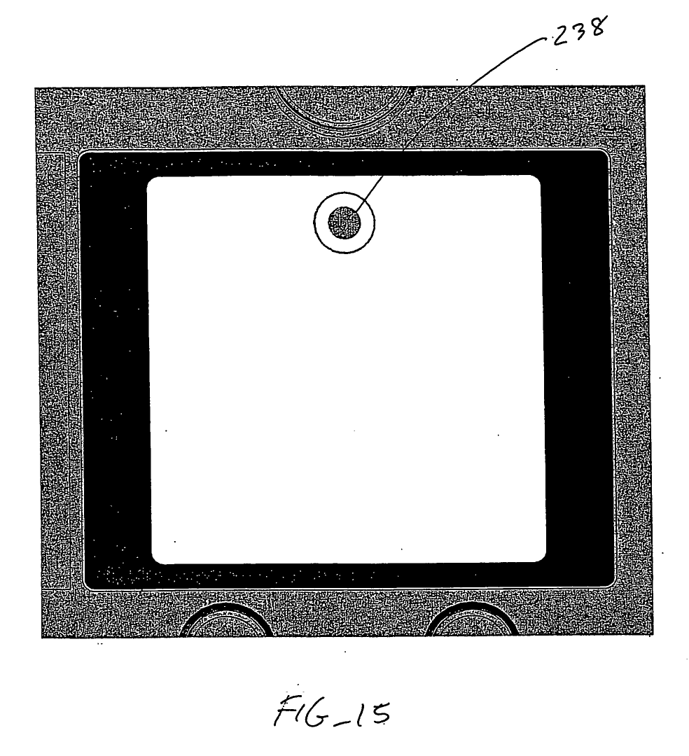

Referring now to FIG. 13, a still further embodiment of the present invention will be described. A display 240 is shown and this display may be positioned anywhere on a housing, such as but not limited to a housing 200. The display 240 may be positioned closer to the center of the housing 200. It may placed on a housing having a circular, square, cylindrical, hexagonal, triangular, oval, ergonomically curved-to-fit-the-hand shape, or polygonal shape. The housing may be made of more than one material, such as a rubber bottom surface, rubber bottom half, or rubber edges to facilitate handling an grip by the user. The housing may be textured, have ribs, or other contour to ease the handling.

As seen in FIG. 13, lancing setting 241 may be appear on the display 240 to assist the user in determining how the lancing device is set to operate. In one embodiment, the numeral displayed may be a numerical representation of lancing depth. The number may be on a scale of some sort, such as in one embodiment, between the depths of 0.0 to 9.9. In another nonlimiting example, the setting 241 may represent the speed setting of the lancet or penetrating member. The speed setting may be selected, in one embodiment, for an inbound path and an outbound path.

Of course, other lancing parameters as discussed herein or in the referenced applications may be represented by the settings 241. For example, the number at the top left hand side may represent the number of penetrating members remaining. A battery strength indicator may also be included. The display 240 may show a variety of information, including but not limited to, the number of target tissue penetrating events, time and date of the last selected number of target tissue penetrating events, time interval between alarm and target tissue penetrating event, stratum corneum thickness when target tissue is below the skin surface and underlying tissue, time of day, energy consumed by penetrating member driver to drive penetrating member into target tissue, depth of penetrating member penetration, velocity of penetrating member, a desired velocity profile, velocity of penetrating member into target tissue, velocity of penetrating member out of target tissue, dwell time of penetrating member in target tissue, a target tissue relaxation parameter, force delivered on target tissue, dwell time of penetrating member, battery status of tissue penetrating system and its components, tissue penetrating system status, consumed energy, speed profile of penetrating member as it advances through target tissue, a target tissue relaxation parameter, information relative to contact of a penetrating member with target tissue before penetration by penetrating member, information relative to a change of speed of penetrating member as in travels in target tissue, information relative to consumed sensors, information relative to consumed penetrating members.

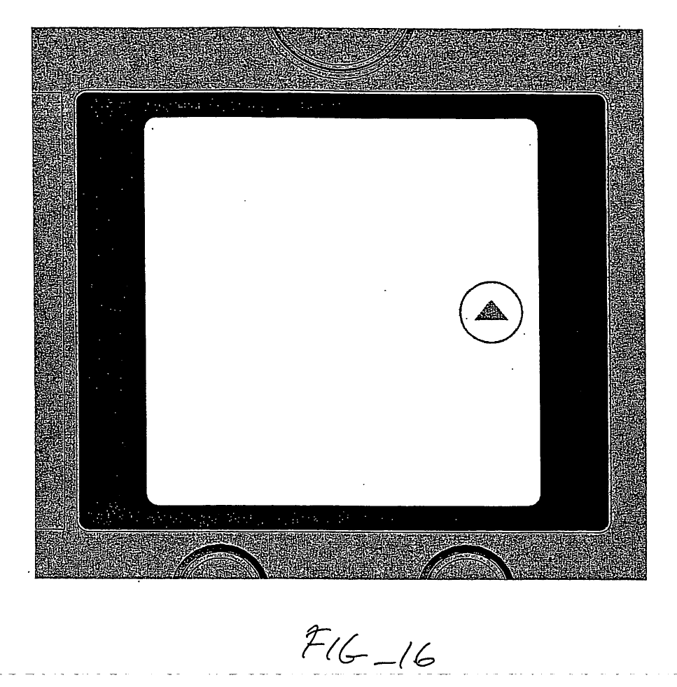

Referring now to FIG. 14, embodiments of the present invention may have a display 240 that includes a screen saver mode. In the present nonlimiting example, the screen saver is a circle 242 that may move about on the screen. In other embodiments, the screen saver includes a plurality of bars 244 that moves in a circular pattern as indicated by arrow 246. The bars 244 may of course move in other paths besides circular, such as but not limited to figure-8, triangular, square, etc . . . . Other screensaver patterns as known in the art such as spacewarp or others as seen in many personal computer monitor displays may also be adapted for use with the present display 240. In one nonlimiting example, this screensaver may come on after 30 seconds, 45 seconds, or 60 seconds of non-use by the user. As soon as a user touches a button or other input device, the screensaver will disappear and the display 240 will return to normal operating mode. The display 240 may also be a touch sensitive display as known in the art. In some embodiments, the entire display may go inactive after the screensaver has been displayed for certain period of time and there has been no user activity. Such a time period may be preset or it may be set by the user.

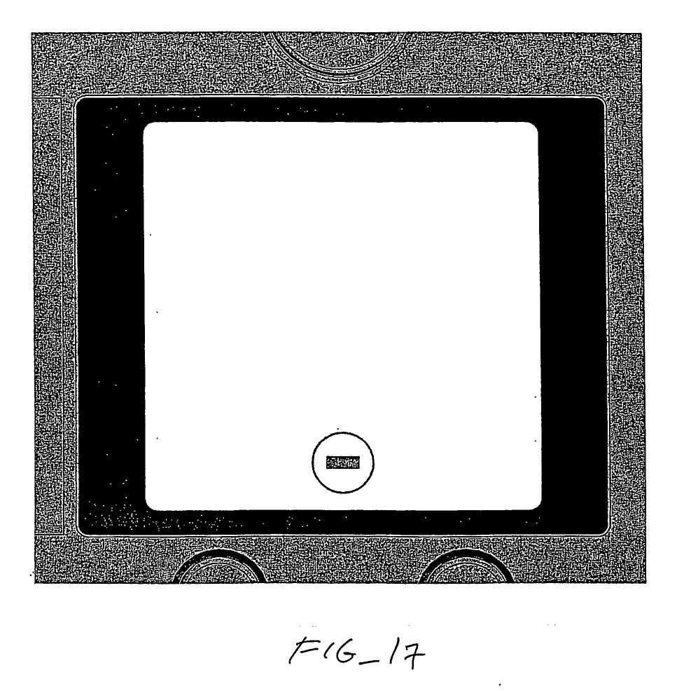

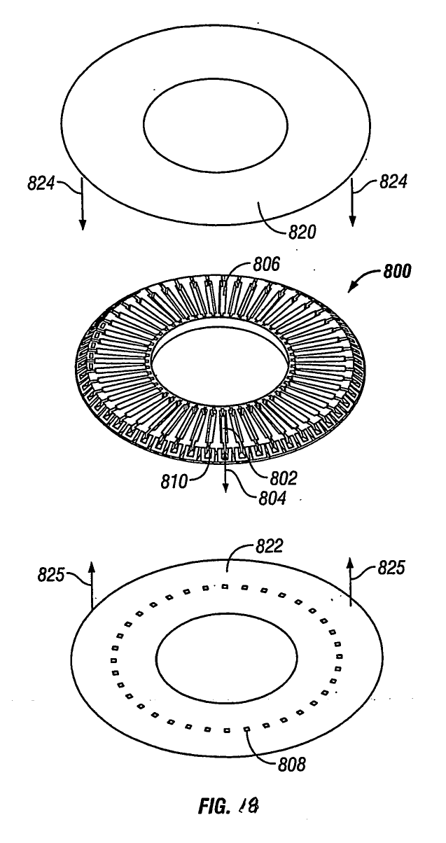

FIGS. 15, 16, and 17 show that icons may be displayed individually on the display 240 to focus the users attention on the task at hand. As a nonlimiting example, the indicator 238 in FIG. 15 may appear, flash, or animate to show the it is time for the lancet to be actuated. FIG. 16 shows that it is time to move the slider. FIG. 17 shows that it is time to adjust the lancet setting. Any of the icons shown in the above figures may be used or displayed singly or in any combination, multiple combination, or in some sequence to provide information to the user. As previously mentioned, the appearance of the icon or logos for any of the embodiments described herein may correspond in position to text or indicators on the housing of the device. The icons displayed may be but are not limited to, filled or empty circles, filled or empty squares, triangles, arrows, lines, emoticons, smiley faces, faces, sad faces, text, a sequence of symbols, droplets, stop signs, traffic signal type icons, hexagons, polygons, colored symbols or icons, or any single or multiple combination of the above. Any of the above may be filled or unfilled icons, symbols, or logos and may move when being displayed on screen 240. They may also change states, such as color, being filled or unfilled, display time to completion, or a count-down timer using numbers or symbols or any single or multiple combination of the above.

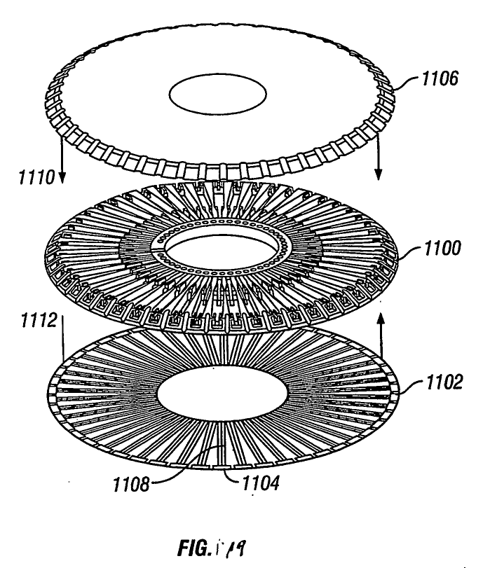

FIG. 18 shows one embodiment of a cartridge 800 which may be removably inserted into an apparatus for driving penetrating members to pierce skin or other tissue. It should be understood that the display 240 may be used with tissue penetrating devices that use a plurality of penetrating members, such as one using a disc shown in FIG. 18. It should also be understood that the display 240 may also be adapted for use with tissue penetrating devices that do not contain multiple penetrating members.

Referring now to FIG. 18, the cartridge 800 has a plurality of penetrating members 802 that may be individually or otherwise selectively actuated so that the penetrating members 802 may extend outward from the cartridge, as indicated by arrow 804, to penetrate tissue. In the present embodiment, the cartridge 800 may be based on a flat disc with a number of penetrating members such as, but in no way limited to, (25, 50, 75, 100, . . . ) arranged radially on the disc or cartridge 800. It should be understood that although the cartridge 800 is shown as a disc or a disc-shaped housing, other shapes or configurations of the cartridge may also work without departing from the spirit of the present invention of placing a plurality of penetrating members to be engaged by a penetrating member driver.

Each penetrating member 802 may be contained in a molded cavity 806 in the cartridge 800 with the penetrating member's sharpened end facing radially outward and may be in the same plane as that of the cartridge. Although not limited in this manner, the ends of the protective cavities 806 may be divided into individual fingers (such as one for each cavity) on the outer periphery of the disc. The particular shape of each cavity 806 may be designed to suit the size or shape of the penetrating member therein or the amount of space desired for placement of the analyte detecting members 808. For example and not limitation, the cavity 806 may have a V-shaped cross-section, a U-shaped cross-section, C-shaped cross-section, a multi-level cross section or the other cross-sections. The opening 810 through which a penetrating member 802 may exit to penetrate tissue may also have a variety of shapes, such as but not limited to, a circular opening, a square or rectangular opening, a U-shaped opening, a narrow opening that only allows the penetrating member to pass, an opening with more clearance on the sides, a slit, or the other shapes.

After actuation, the penetrating member 802 is returned into the cartridge and may be held within the cartridge 800 in a manner so that it is not able to be used again. By way of example and not limitation, a used penetrating member may be returned into the cartridge and held by the launcher in position until the next lancing event. At the time of the next lancing, the launcher may disengage the used penetrating member with the cartridge 800 turned or indexed to the next clean penetrating member such that the cavity holding the used penetrating member is position so that it is not accessible to the user (i.e. turn away from a penetrating member exit opening). In some embodiments, the tip of a used penetrating member may be driven into a protective stop that hold the penetrating member in place after use. The cartridge 800 is replaceable with a new cartridge 800 once all the penetrating members have been used or at such other time or condition as deemed desirable by the user.

Referring still to FIG. 18, the cartridge 800 may provide sterile environments for penetrating members via seals, foils, covers, polymeric, or similar materials used to seal the cavities and provide enclosed areas for the penetrating members to rest in. In the present embodiment, a foil or seal layer 820 is applied to one surface of the cartridge 800. The seal layer 820 may be made of a variety of materials such as a metallic foil or other seal materials and may be of a tensile strength and other quality that may provide a sealed, sterile environment until the seal layer 820 is penetrate by a suitable or penetrating device providing a preselected or selected amount of force to open the sealed, sterile environment. Each cavity 806 may be individually sealed with a layer 820 in a manner such that the opening of one cavity does not interfere with the sterility in an adjacent or other cavity in the cartridge 800. As seen in the embodiment of FIG. 18, the seal layer 820 may be a planar material that is adhered to a top surface of the cartridge 800.

Depending on the orientation of the cartridge 800 in the penetrating member driver apparatus, the seal layer 820 may be on the top surface, side surface, bottom surface, or other positioned surface. For ease of illustration and discussion of the embodiment of FIG. 18, the layer 820 is placed on a top surface of the cartridge 800. The cavities 806 holding the penetrating members 802 are sealed on by the foil layer 820 and thus create the sterile environments for the penetrating members. The foil layer 820 may seal a plurality of cavities 806 or only a select number of cavities as desired.

In a still further feature of FIG. 18, the cartridge 800 may optionally include a plurality of analyte detecting members 808 on a substrate 822 which may be attached to a bottom surface of the cartridge 800. It should be understood that the display may be adapted for use with cartridges with and without analyte detecting members 808. The substrate may be made of a material such as, but not limited to, a polymer, a foil, or other material suitable for attaching to a cartridge and holding the detecting members 808. As seen in FIG. 18, the substrate 822 may hold a plurality of analyte detecting members, such as but not limited to, about 10-50, 50-100, or other combinations of analyte detecting members. This facilitates the assembly and integration of analyte detecting members 808 with cartridge 800. These analyte detecting members 808 may enable an integrated body fluid sampling system where the penetrating members 802 create a wound tract in a target tissue, which expresses body fluid that flows into the cartridge for analyte detection by at least one of the analyte detecting members 808. The substrate 822 may contain any number of analyte detecting members 808 suitable for detecting analytes in cartridge having a plurality of cavities 806. In one embodiment, many analyte detecting members 808 may be printed onto a single substrate 822 which is then adhered to the cartridge to facilitate manufacturing and simplify assembly. The analyte detecting members 808 may be electrochemical in nature. The analyte detecting members 808 may further contain enzymes, dyes, or other detectors which react when exposed to the desired analyte. Additionally, the analyte detecting members 808 may comprise of clear optical windows that allow light to pass into the body fluid for analyte analysis. The number, location, and type of analyte detecting member 808 may be varied as desired, based in part on the design of the cartridge, number of analytes to be measured, the need for analyte detecting member calibration, and the sensitivity of the analyte detecting members. If the cartridge 800 uses a analyte detecting member arrangement where the analyte detecting members are on a substrate attached to the bottom of the cartridge, there may be through holes (as shown in FIG. 76), wicking elements, capillary tube or other devices on the cartridge 800 to allow body fluid to flow from the cartridge to the analyte detecting members 808 for analysis. In other configurations, the analyte detecting members 808 may be printed, formed, or otherwise located directly in the cavities housing the penetrating members 802 or areas on the cartridge surface that receive blood after lancing.

The use of the seal layer 820 and substrate or analyte detecting member layer 822 may facilitate the manufacture of these cartridges 10. For example, a single seal layer 820 may be adhered, attached, or otherwise coupled to the cartridge 800 as indicated by arrows 824 to seal many of the cavities 806 at one time. A sheet 822 of analyte detecting members may also be adhered, attached, or otherwise coupled to the cartridge 800 as indicated by arrows 825 to provide many analyte detecting members on the cartridge at one time. During manufacturing of one embodiment of the present invention, the cartridge 800 may be loaded with penetrating members 802, sealed with layer 820 and a temporary layer (not shown) on the bottom where substrate 822 would later go, to provide a sealed environment for the penetrating members. This assembly with the temporary bottom layer is then taken to be sterilized. After sterilization, the assembly is taken to a clean room where the temporary bottom layer is removed and the substrate 822 with analyte detecting members is coupled to the cartridge as shown in FIG. 18. This process allows for the sterile assembly of the cartridge with the penetrating members 802 using processes and/or temperatures that may degrade the accuracy or functionality of the analyte detecting members on substrate 822.

In some embodiments, more than one seal layer 820 may be used to seal the cavities 806. As examples of some embodiments, multiple layers may be placed over each cavity 806, half or some selected portion of the cavities may be sealed with one layer with the other half or selected portion of the cavities sealed with another sheet or layer, different shaped cavities may use different seal layer, or the like. The seal layer 820 may have different physical properties, such as those covering the penetrating members 802 near the end of the cartridge may have a different color such as red to indicate to the user (if visually inspectable) that the user is down to say 10, 5, or other number of penetrating members before the cartridge should be changed out.

Referring now to FIG. 19, a still further embodiment of the present invention will now be described. FIG. 19 is an exploded view showing a cartridge 1100, a layer 1102 with a plurality of analyte detecting member 1104, and a sterility barrier 1106. The analyte detecting members 1104 on layer 1102 may have leads or connectors 1108 that extend along the layer 1102. In some embodiments, these leads 1108 extend all the way to the inner circumference of the layer 1102. In other embodiments, the leads 1108 may not extend all the way to the inner circumference. As indicated by arrows 1110 and 1112, the layer 1102 and sterility barrier 1106 may be coupled to the cartridge 1100 to form a device for use with a lancing apparatus 880. In most embodiments, penetrating members (not shown) are contained in the cartridge 1100 prior to coupling the sterility barrier 1106 to the cartridge 1100.

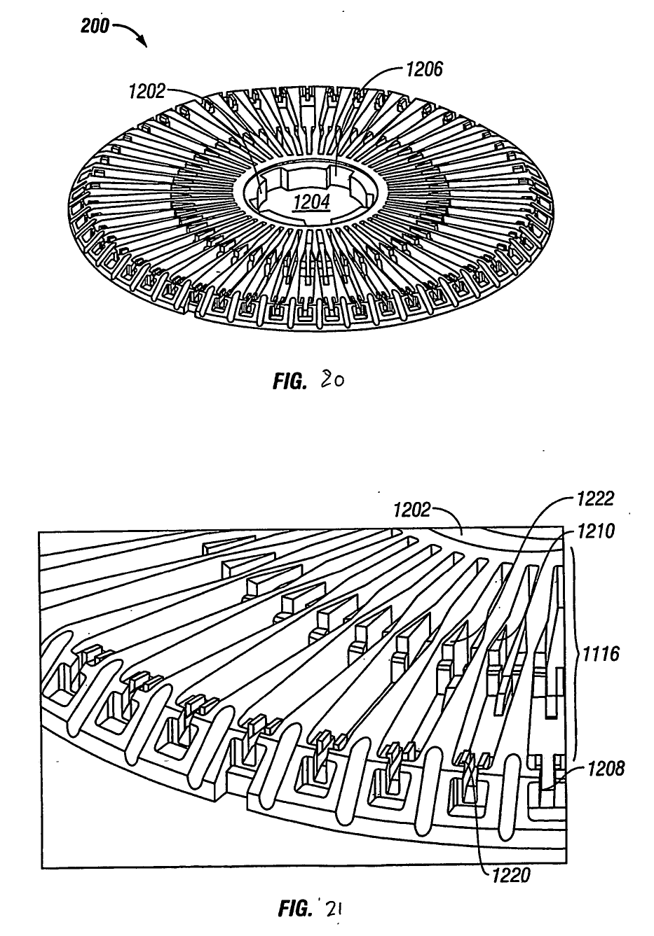

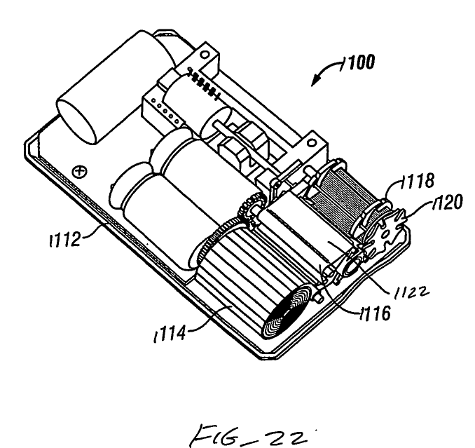

Referring now to FIGS. 20 and 21, a still further embodiment of a cartridge according to the present invention will now be described. The cartridge 1200 of FIG. 20 includes a plurality of notches 1202 formed in an opening 1204 in the cartridge. These notches 1202 may be used for a variety of purpose, including but not limited to, positioning of the cartridge 1200 in a lancing apparatus or for rotation purposes to change position of cavities 1116 aligned with a penetrating member launching device. The hub (not shown) which would mate with the opening 1204 may be rotating device that will be used to control which cavity 1116 and penetrating member is positioned for engagement with the launcher.

In one embodiment, the cartridge 1200 may include front bearing areas 1208 for guiding a penetrating member and rear bearing areas 1210. The rear bearing areas 1210 may be a length sufficient so that the penetrating member may create a wound in the target tissue without losing contact or guidance from the rear bearing area 1210. This provides for more control of the cutting path taken by the penetrating member. The cavity provides sufficient open space for a penetrating member gripper to accommodate the throw distance used by the gripper to advance the penetrating member to contact tissue. In some embodiments, a middle guide bearing 1212 may be used. In such an embodiment, the gripper would grip a rear portion of the penetrating member, with both bearings remaining in “front” of the gripper, and the throw area of cavity 1116 moved towards at least the rear half (in one embodiment) of the cartridge. As a nonlimiting example, the throw distance may be adjusted as desired to take up more than ½ of cavity 1116, less than ⅓, or less than ¼ of the cavity. A narrowed portion 1218 may be included to hold the penetrating members when the penetrating members are not being actuated.

As seen in FIG. 21, the portion 1220 on the cartridge 1200 may be open or pressed to close the top surface of the front bearing (while still having an opening allowing the penetrating member to pass). There rear of cavity 1116 may be narrowed to hold the penetrating member in place. Portions 1222 may also be used to deal with flash associated with the manufacturing process.

FIG. 22 shows a still further embodiment of penetrating member delivery devices. From this view, one can see an embodiment of the electromechanical penetrating member driver 1110, capacitors 1112, a spool 1114 of tape 1122 having unused penetrating members, a tape peeling roller 1116, a penetrating member drive chuck 1118, and a used penetrating member drum 1120. The tape 1122 may be wound into a spool 1114 with the tape being fed towards a first roller 1124. Tape peeling rollers 1116 and 1126 collect the used tape after the tape is peeled and penetrating member removed. In one embodiment, the tape 1122 may comprise of a bottom layer and a top layer with the unused penetrating members held therebetween. The tape 1122 may fully enclose each of the penetrating member. In other embodiments, the tape 1122 only partially covers each penetrating member and may cover those portions that penetrate the patient and are to remain sterile prior to use. This embodiment is shown to teach that the present display may be used with a variety of technologies and techniques for providing multiple penetrating members in a device. The display 240 is not limited to the technique used to load the penetrating member for use.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, the location of the penetrating member drive device may be varied, relative to the penetrating members or the cartridge. With any of the above embodiments, the penetrating member tips may be uncovered during actuation (i.e. penetrating members do not pierce the penetrating member enclosure or protective foil during launch). With any of the above embodiments, the penetrating members may be a bare penetrating member during launch. With any of the above embodiments, the penetrating members may be bare penetrating members prior to launch as this may allow for significantly tighter densities of penetrating members. In some embodiments, the penetrating members may be bent, curved, textured, shaped, or otherwise treated at a proximal end or area to facilitate handling by an actuator. The penetrating member may be configured to have a notch or groove to facilitate coupling to a gripper. The notch or groove may be formed along an elongate portion of the penetrating member. With any of the above embodiments, the cavity may be on the bottom or the top of the cartridge, with the gripper on the other side. In some embodiments, analyte detecting members may be printed on the top, bottom, or side of the cavities. The front end of the cartridge maybe in contact with a user during lancing. The same driver may be used for advancing and retraction of the penetrating member. The penetrating member may have a diameters and length suitable for obtaining the blood volumes described herein. The penetrating member driver may also be in substantially the same plane as the cartridge. The driver may use a through hole or other opening to engage a proximal end of a penetrating member to actuate the penetrating member along a path into and out of the tissue.

Any of the features described in this application or any reference disclosed herein may be adapted for use with any embodiment of the present invention. For example, the devices of the present invention may also be combined for use with injection penetrating members or needles as described in commonly assigned, copending U.S. patent application Ser. No. 10/127,395 (Attorney Docket No. 38187-2551) filed Apr. 19, 2002. An analyte detecting member to detect the presence of foil may also be included in the lancing apparatus. For example, if a cavity has been used before, the foil or sterility barrier will be punched. The analyte detecting member can detect if the cavity is fresh or not based on the status of the barrier. It should be understood that in optional embodiments, the sterility barrier may be designed to pierce a sterility barrier of thickness that does not dull a tip of the penetrating member. The lancing apparatus may also use improved drive mechanisms. For example, a solenoid force generator may be improved to try to increase the amount of force the solenoid can generate for a given current. A solenoid for use with the present invention may have five coils and in the present embodiment the slug is roughly the size of two coils. One change is to increase the thickness of the outer metal shell or windings surround the coils. By increasing the thickness, the flux will also be increased. The slug may be split; two smaller slugs may also be used and offset by ½ of a coil pitch. This allows more slugs to be approaching a coil where it could be accelerated. This creates more events where a slug is approaching a coil, creating a more efficient system.

In another optional alternative embodiment, a gripper in the inner end of the protective cavity may hold the penetrating member during shipment and after use, eliminating the feature of using the foil, protective end, or other part to retain the used penetrating member. Some other advantages of the disclosed embodiments and features of additional embodiments include: same mechanism for transferring the used penetrating members to a storage area; a high number of penetrating members such as 25, 50, 75, 100, 500, or more penetrating members may be put on a disk or cartridge; molded body about a lancet becomes unnecessary; manufacturing of multiple penetrating member devices is simplified through the use of cartridges; handling is possible of bare rods metal wires, without any additional structural features, to actuate them into tissue; maintaining extreme (better than 50 micron—lateral—and better than 20 micron vertical) precision in guiding; and storage system for new and used penetrating members, with individual cavities/slots is provided. The housing of the lancing device may also be sized to be ergonomically pleasing. In one embodiment, the device has a width of about 56 mm, a length of about 105 mm and a thickness of about 15 mm. Additionally, some embodiments of the present invention may be used with non-electrical force generators or drive mechanism. For example, the punch device and methods for releasing the penetrating members from sterile enclosures could be adapted for use with spring based launchers. The gripper using a frictional coupling may also be adapted for use with other drive technologies. With any of the embodiments described herein, it should be understood that the display may be used with lancing only devices or it may be used with fully integrated devices which will penetrate tissue, obtain a fluid sample, and provide an analyte measurement. In any of the embodiments, an appropriate processor may be coupled to the display to control when a symbol should be displayed, the sequence, and the like. In some embodiments, memory or other storage device may also be coupled to the display or the processor to store information to be shown on the display.

Still further optional features may be included with the present invention. For example, with any of the above embodiments, the location of the penetrating member drive device may be varied, relative to the penetrating members or the cartridge. With any of the above embodiments, the penetrating member tips may be uncovered during actuation (i.e. penetrating members do not pierce the penetrating member enclosure or protective foil during launch). The penetrating members may be a bare penetrating member during launch. The same driver may be used for advancing and retraction of the penetrating member. Different analyte detecting members detecting different ranges of glucose concentration, different analytes, or the like may be combined for use with each penetrating member. Non-potentiometric measurement techniques may also be used for analyte detection. For example, direct electron transfer of glucose oxidase molecules adsorbed onto carbon nanotube powder microelectrode may be used to measure glucose levels. In some embodiments, the analyte detecting members may formed to flush with the cartridge so that a “well” is not formed. In some other embodiments, the analyte detecting members may formed to be substantially flush (within 200 microns or 100 microns) with the cartridge surfaces. In all methods, nanoscopic wire growth can be carried out via chemical vapor deposition (CVD). In all of the embodiments of the invention, preferred nanoscopic wires may be nanotubes. Any method useful for depositing a glucose oxidase or other analyte detection material on a nanowire or nanotube may be used with the present invention. Additionally, for some embodiments, any of the cartridge shown above may be configured without any of the penetrating members, so that the cartridge is simply an analyte detecting device. Still further, the indexing of the cartridge may be such that adjacent cavities may not necessarily be used serially or sequentially. As a nonlimiting example, every second cavity may be used sequentially, which means that the cartridge will go through two rotations before every or substantially all of the cavities are used. As another nonlimiting example, a cavity that is 3 cavities away, 4 cavities away, or N cavities away may be the next one used. This may allow for greater separation between cavities containing penetrating members that were just used and a fresh penetrating member to be used next. This application cross-references commonly assigned copending U.S. patent applications Ser. No. 10/323,622 (Attorney Docket No. 38187-2606) filed Dec. 18, 2002; commonly assigned copending U.S. patent applications Ser. No. 10/323,623 (Attorney Docket No. 38187-2607) filed Dec. 18, 2002; and commonly assigned copending U.S. patent applications Ser. No. 10/323,624 (Attorney Docket No. 38187-2608) filed Dec. 18, 2002. This application is also related to commonly assigned copending U.S. patent applications Ser. Nos. 10/335,142, 10/335,215, 10/335,258, 10/335,099, 10/335,219, 10/335,052, 10/335,073, 10/335,220, 10/335,252, 10/335,218, 10/335,211, 10/335,257, 10/335,217, 10/335,212, 10/335,241, 10/335,183, 10/335,082, 10/335,240, 10/335,259, 10/335,182 (Attorney Docket No. 38187-2633 through 38187-2652) filed Dec. 31, 2002. All applications listed above are fully incorporated herein by reference for all purposes. Expected variations or differences in the results are contemplated in accordance with the objects and practices of the present invention. It is intended, therefore, that the invention be defined by the scope of the claims which follow and that such claims be interpreted as broadly as is reasonable.

1. (canceled)

2. A tissue penetrating system, comprising:
a housing;
at least one penetrating member positioned in the housing,
a visual display on said housing, said display configured to show lancing parameters for the penetrating member.

3. The tissue penetrating system of claim 2 wherein the display shows at least one of the following:
a number of penetrating members used, number of target tissue penetrating events, time and date of the last selected number of target tissue penetrating events, time interval between alarm and target tissue penetrating event, stratum corneum thickness, time of day, energy consumed by a penetrating member driver to drive a penetrating member into the target tissue, depth of penetrating member penetration, velocity of the penetrating member, a desired velocity profile, velocity of the penetrating member into the target tissue, velocity of the penetrating member out of the target tissue, dwell time of the penetrating member in the target tissue, a target tissue relaxation parameter, force delivered on the target tissue, dwell time of the penetrating member, battery status, system status, consumed energy, speed profile of the penetrating member as the penetrating penetrates and advances through the target tissue, a tissue target tissue relaxation parameter, information relative to contact of a penetrating member with target tissue before penetration by the penetrating member, information relative to a change of speed of a penetrating member as in travels in the target tissue, information relative to consumed sensors, or information relative to consumed penetrating members.

4. The tissue penetrating system of claim 2 wherein said visual display has at least one visual indicator position next to a corresponding marking on the housing.

5. The tissue penetrating system of claim 2 wherein said visual display has at least one visual indicator directing the user to take particular action.

6. The tissue penetrating system of claim 2 further comprising:
a tissue pressure applicator coupled to the housing;
wherein said visual display has at least one visual indicator position next to a corresponding marking on the housing.

7. A tissue penetrating system, comprising:
a housing;
a penetrating members positioned in the housing,
an analyte detecting member coupled to a sample chamber, the analyte detecting member being configured to determine a concentration of an analyte in a body fluid using a sample of less than 1 μL of a body fluid disposed in the sample chamber;
a visual display on said housing, said visual display having a screen saver which is activated after a preset period of nonuse by a user.

8. The tissue penetrating system of claim 7 wherein the analyte detecting member being configured to determine a concentration of an analyte in a body fluid using a sample of less than 500 μL of a body fluid disposed in the sample chamber.

9. The tissue penetrating system of claim 7 wherein the analyte detecting member being configured to determine a concentration of an analyte in a body fluid using a sample of less than 100 μL of a body fluid disposed in the sample chamber.

10. A tissue penetrating system, comprising:
a housing;
a penetrating members positioned in the housing,
a visual display on said housing, said visual display having at lease one visual indicator position next to a corresponding marking on the housing;
at least one button on said housing for changing lancet settings shown on the visual display.

11. The tissue penetrating system of claim 10 further comprising a plurality of buttons for changing device settings.

12. The tissue penetrating system of claim 10 further comprising a processor configured to control the display to show the desired symbol.

13. The tissue penetrating system of claim 10 further comprising memory coupled to said display for storing information to be displayed.

14. The tissue penetrating system of claim 10 further comprising a processor configured to control the display to show a desired symbol when it is time for the user to launch the penetrating member.

15. The tissue penetrating system of claim 10 further comprising a processor configured to control the display to show a desired symbol when it is time for the user to load the next penetrating member.

16. The tissue penetrating system of claim 10 further comprising a processor configured to control the display to show a desired symbol when it is time for the user adjust the lancing depth.

17. The tissue penetrating system of claim 10 further comprising a processor configured to control the display to show a desired symbol when it is time for the user adjust the lancing speed.

18. The tissue penetrating system of claim 10 further comprising a processor configured to control the display to show a desired symbol when it is time for the user to replace a cartridge holding a plurality of penetrating members.

19. The tissue penetrating system of claim 10 further comprising a processor configured to control the display to show a desired symbol when it is time for the user to set default device parameters.

20. The tissue penetrating system of claim 10 further comprising a processor configured to control the display to show a desired symbol when it is time for the user to replace a battery in the system.
